(19) United States
(12) Patent Application Publication
(10) Pub. No.: US 2020/0323762 A1
(43) Pub. Date: Oct. 15, 2020

(54) SELF-ASSEMBLY TYPE JANUS MICROPARTICLE AND PREPARATION METHOD THEREFOR

(71) Applicant: AMOREPACIFIC CORPORATION, Seoul (KR)

(72) Inventors: Hyunsuk LEE, Yongin-si, Gyeonggi-do (KR); Yan LI, Yongin-si, Gyeonggi-do (KR); Yongjin KIM, Yongin-si, Gyeonggi-do (KR); Johnhwan LEE, Yongin-si, Gyeonggi-do (KR); Kyungho CHOI, Yongin-si, Gyeonggi-do (KR); Jinwoong KIM, Seongnam-si, Gyeonggi-do (KR); Jaehong CHO, Seoul (KR); Jeong Won KIM, Seoul (KR); Youngbok LEE, Seoul (KR); Bum Jun PARK, Suwon-si, Gyeonggi-do (KR); Jangwoo CHO, Suwon-si, Gyeonggi-do (KR)

(21) Appl. No.: 16/303,432

(22) PCT Filed: May 24, 2016
(86) PCT No.: PCT/KR2016/005487

§ 371 Date: Nov. 20, 2018

(51) Int. Cl. A61K 8/81; A61K 8/06; A61K 8/37

(52) U.S. Cl. CPC A61K 8/8117; A61K 8/064; A61K 2800/654; A61K 2800/10; A61K 8/37

(57) ABSTRACT

The present invention relates to self-assembly type Janus microparticles and a preparation method therefor. The present invention can be used for preparing Janus microparticles in which a degree of phase separation is precisely controlled, for realizing stable interfacial orientation by selectively coating only one side of particles with a metal oxide, and for mass-producing Janus microparticles which have a degree of phase separation precisely controlled and exhibit extreme amphiphilicity in an anisotropic structure. Therefore, the Janus microparticles and the preparation method therefor according to the present invention can be widely used in various fields, thus contributing to industrial development.

### TECHNICAL FIELD

The present disclosure relates to a self-assembly type Janus microparticle and a method for preparing the same.

### BACKGROUND ART

In cosmetic and pharmaceutical fields, development of a formulation that can stably encapsulate substances effective for skin to ensure their effective action on the skin and improve the skin condition. However, many physiologically active substances are hardly soluble or unstable in aqueous phase and make the entire system unstable.

Techniques for encapsulating those substances more stably and easily in a formulation have been developed to overcome this. Representative examples include emulsion particles formed by treating a semi-formulation prepared using a surfactant having a particular hydrophilic/hydrophobic ratio with a high-pressure emulsification apparatus, etc., liposomes wherein active ingredients are encapsulated by forming one or more layers using phospholipids derived from plants or animals, and so forth.

In addition, researches are being conducted on Pickering emulsions which can form stabilized macroemulsion particles using micro-sized fine particles. The fine particles in the Pickering emulsion exhibit different surface contact angle between the aqueous phase and the oil phase depending on the properties of the particles, and either oil/water or water/oil macroemulsion particles are formed depending on the contact angle.

Although researches are being conducted on fine particles that can be widely used such as Pickering emulsions, etc., actual application was not easy owing to the problems of limited control of the morphology of the fine particles, indefinite amphiphilicity, limited ability of maintaining macroemulsion particles, difficulty in mass production, etc.

### REFERENCES OF RELATED ART

### Patent Documents

Korean Patent Publication No. 10-1997-0025588 (Jun. 24, 1997).

### DISCLOSURE

### Technical Problem

In an aspect, the present disclosure is directed to providing a self-assembly type Janus microparticle wherein clear phase separation is achieved and a method for preparing the same in order to allow for free control of the morphology of the Janus microparticle, improvement in emulsion drop retention time and mass production of uniform Janus particles.

### Technical Solution

In an aspect, the present disclosure provides a Janus microparticle containing: a first domain containing polystyrene; and a second domain containing poly(tetradecyl acrylate).

In another aspect, the present disclosure provides an emulsion composition containing the Janus microparticle and a cosmetic composition containing the emulsion composition.

In another aspect, the present disclosure provides the method for preparing a Janus microparticle and a method for controlling the structure of an amphiphilic microparticle.

### Advantageous Effects

In an aspect, the present disclosure provides a Janus microparticle wherein clear phase separation is achieved and a method for preparing the same. The amphiphilic Janus microparticle is applicable to various fields and can be produced in large scale. In addition, the present disclosure provides a method for controlling the structure of the particle, such that the degree of phase separation of the Janus microparticle can be controlled precisely depending on the purpose and use of the particle.

### BRIEF DESCRIPTION OF DRAWINGS

FIG. 1 schematically shows a process of preparing a Janus microparticle according to an aspect of the present disclosure.

FIG. 2 sequentially shows polystyrene synthesized according to an aspect of the present disclosure, the polystyrene in which a tetradecyl acrylate monomer is swollen and a Janus microparticle in which poly(tetradecyl acrylate) is formed by photopolymerization and phase-separated.

FIG. 3 shows a process of coating a silica nanoparticle on a Janus microparticle according to an aspect of the present disclosure.

FIG. 4 shows Janus microparticles on which a silica particle with a diameter of 100 nm and a silica particle with a diameter of 300 nm are coated according to an aspect of the present disclosure.

FIG. 5 shows that an amphiphilic Janus particle according to an aspect of the present disclosure effectively exhibits wettability on a compatible liquid.

FIG. 6 shows a fluorescence microscopic image showing the polystyrene portion and the poly(tetradecyl acrylate) portion of a Janus microparticle according to an aspect of the present disclosure and an electron microscopic image showing that the Janus microparticle is spherical.

FIG. 7 compares the size of polystyrene and Janus microparticles according to an aspect of the present disclosure.

FIG. 8 shows a result of X-ray photoelectron spectroscopy (XPS) analysis showing that polyvinylpyrrolidone is covalently bonded on the surface of polystyrene according to an aspect of the present disclosure.

FIG. 9a, FIG. 9b, FIG. 9c and FIG. 9d show images of particles prepared by varying the alkyl chain length of alkyl acrylates according to an aspect of the present disclosure and FIG. 9e, FIG. 9g and FIG. 9h show a result of varying the ethanol/water volume ratio in an ethanol/water mixture solvent.

FIGS. 10a-10c show Janus microparticles with different degrees of Janusity prepared by controlling swelling ratio according to an aspect of the present disclosure.

FIGS. 11a and 11b show microscopic images of Pickering emulsions according to an aspect of the present disclosure.

FIG. 12 shows a relationship between the degree of Janusity and the interfacial contact angle of a Janus microparticle according to an aspect of the present disclosure.

FIG. 13 shows a relationship between the degree of Janusity and the retention time of a Pickering emulsion drop according to an aspect of the present disclosure.

### BEST MODE

Hereinafter, the present disclosure is described in detail

In the present disclosure, a Janus microparticle refers to a micrometer-sized particle that has two portions with different structures or properties. Narrowly, it means a spherical particle wherein different portions have different structures or properties. In general, the difference in the structures or properties is derived from the difference in internal or surface structures, bonding or physical or chemical properties.

In the present disclosure, a hydrophilicity-inducing group refers to a group which is capable of forming a bond (including a covalent bond) on the surface of polystyrene, thereby bonding (including hydrogen bonding) a hydrophilic material to itself and inducing the hydrophilic material to be coated outside of the polystyrene.

In the present disclosure, a diameter refers to an average diameter of particles and includes a diameter calculated for an equivalent sphere which is not a perfect sphere. For example, the diameter of the equivalent sphere may be calculated for an equivalent sphere having the same property as the actual particle, such as a sphere with the same maximum length, a sphere with the same minimum length, a sphere with the same mass, a sphere with the same volume, a sphere with the same surface area, a sphere passing through the same sieve aperture, a sphere with the same precipitation speed, etc. The diameter may be averaged.

In an aspect, the present disclosure provides a Janus microparticle including:

a first domain containing polystyrene; and a second domain containing poly(tetradecyl acrylate).

In an exemplary embodiment, a hydrophilicity-inducing group may be covalently bonded on the surface of the polystyrene of the first domain.

In an exemplary embodiment, the first domain may contain: a core containing polystyrene; and a hydrophilic material coating layer coated on the core.

In an exemplary embodiment, the hydrophilic material coating layer may contain a hydrophilic material bonded to the hydrophilicity-inducing group covalently bonded on the surface of the polystyrene.

In an exemplary embodiment, the hydrophilicity-inducing group may include one or more selected from a group consisting of poly(vinyl alcohol), polyvinylpyrrolidone and poloxamer. Specifically, it may be polyvinylpyrrolidone. The poloxamer may be poloxamer 407 or a poly(ethylene oxide)-poly(propylene oxide)-poly(ethylene oxide) (PEO-PPO-PEO) triblock copolymer.

In an exemplary embodiment, the hydrophilic material may include a silica nanoparticle.

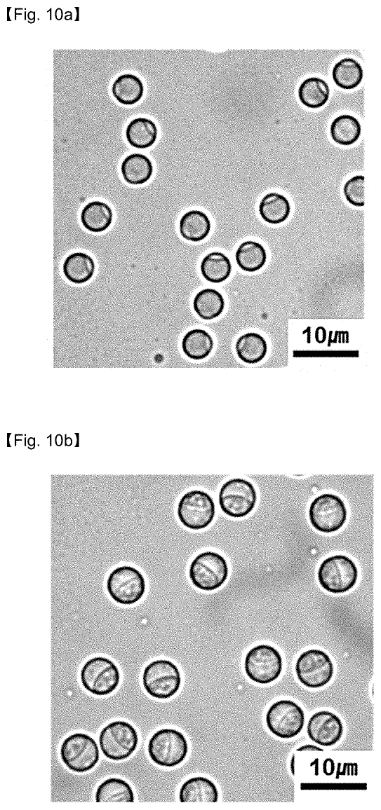

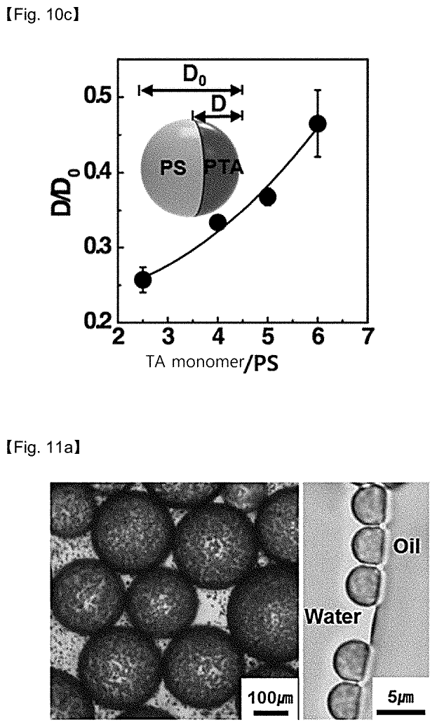

In the present disclosure, a degree of Janusity is defined as D/D₀, where D is the shorter diameter of the second domain of the Janus microparticle and D₀ is the whole diameter of the particle (see FIG. 10c). In an exemplary embodiment, the Janus microparticle may have a degree of Janusity of the second domain with respect to the entire particle of 0.25-0.75. In another exemplary embodiment, the degree of Janusity may be 0.25 or greater, 0.3 or greater, 0.35 or greater, 0.37 or greater, 0.4 or greater, 0.45 or greater, 0.5 or greater, 0.55 or greater, 0.6 or greater or 0.7 or greater or 0.75 or smaller, 0.7 or smaller, 0.6 or smaller, 0.55 or smaller, 0.5 or smaller, 0.45 or smaller, 0.4 or smaller, 0.37 or smaller, 0.35 or smaller or 0.3 or smaller, specifically 0.45-0.55.

In an exemplary embodiment, the Janus microparticle may have a diameter calculated for an equivalent sphere ranging from 1 micrometer (μm) to 100 micrometers. In another exemplary embodiment, the diameter may be 1 μm or greater, 3 μm or greater, 5 μm or greater, 7 μm or greater, 10 μm or greater, 15 μm or greater, 20 μm or greater, 30 μm or greater, 60 μm or greater or 80 μm or greater or 100 μm or smaller, 80 μm or smaller, 60 μm or smaller, 30 μm or smaller, 20 μm or smaller, 15 μm or smaller, 10 μm or smaller, 7 μm or smaller, 5 μm or smaller or 3 μm or smaller, specifically 3-10 μm.

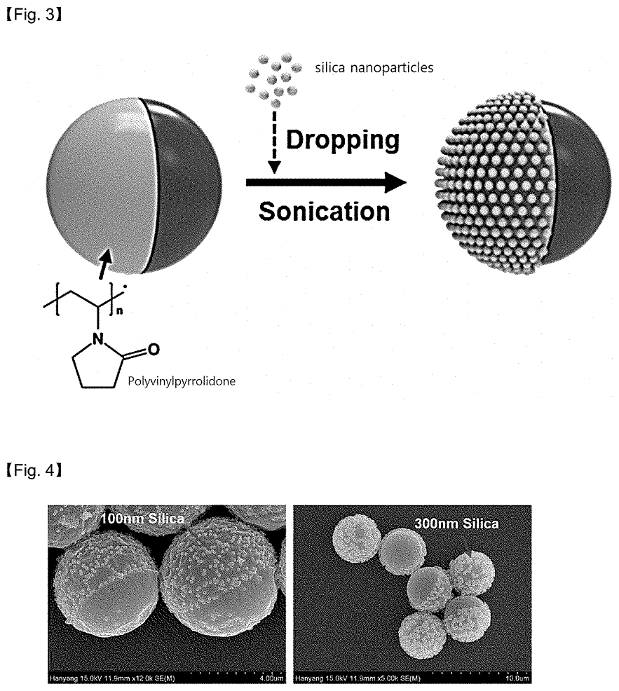

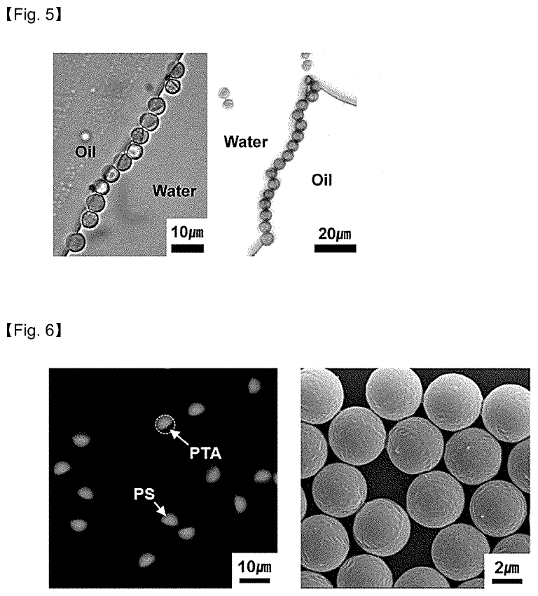

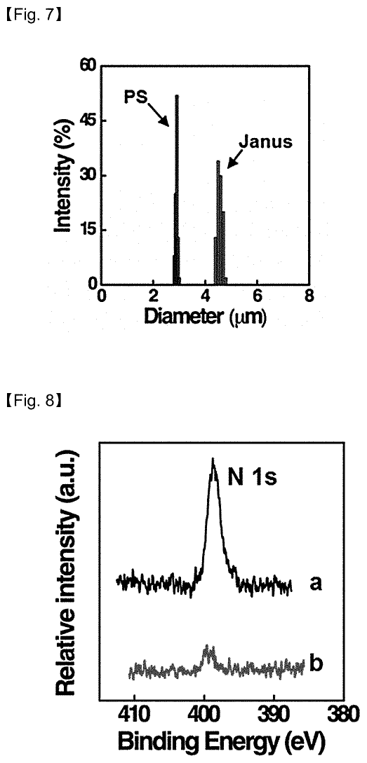

Because clear phase separation is achieved in the Janus microparticle (see FIG. 2, FIG. 3 and FIG. 4), the Janus microparticle can be utilized variously in the fields requiring clear phase separation such as amphiphilicity, etc. In particular, because a hydrophobic portion and a hydrophilic portion are clearly separated when hydrophilicity is conferred by coating a silica nanoparticle (see FIG. 4 and FIG. 5), the Janus microparticle can have remarkably superior property as compared to the existing particle where phase separation was difficult. Also, it may be widely applicable to various uses including a Pickering emulsion owing to the clear amphiphilicity.

In another aspect, the present disclosure provides an emulsion composition containing the Janus microparticle.

In an exemplary embodiment, the emulsion may be a Pickering emulsion.

In an exemplary embodiment, the emulsion may be a water-in-oil (w/o) emulsion when a degree of Janusity of a second domain with respect to the entire Janus microparticle is equal to or greater than 0.25 and smaller than 0.37 and may be an oil-in-water (o/w) emulsion when the degree of Janusity is equal to or greater than 0.37 and smaller than 0.75.

In an exemplary embodiment, the emulsion composition may have improved retention time of an emulsion drop. In another exemplary embodiment, the retention time may be 20 hours or longer, 40 hours or longer, 60 hours or longer, 80 hours or longer or 100 hours or longer, specifically 60 hours or longer.

The existing emulsion composition was difficult in maintaining quality due to the short retention time of an emulsion drop and it was difficult to improve the problem. In contrast, the emulsion composition according to an aspect of the present disclosure has remarkably improved retention time of an emulsion drop because the degree of Janusity is controlled to 0.5 or close thereto (see FIG. 13).

In another aspect, the present disclosure provides a cosmetic composition containing the emulsion composition.

The cosmetic composition according to the present disclosure is not particularly limited in formulation. For example, it may be formulated as a hair tonic, a scalp treatment, a hair cream, an ointment, a softening lotion, an astringent lotion, a nourishing lotion, an eye cream, a nourishing cream, a massage cream, a cleansing cream, a cleansing foam, a cleansing water, a powder, an essence, a pack, a body lotion, a body cream, a body oil, a body essence, a makeup base, a foundation, a hairdye, a shampoo, a rinse, a body cleanser, a toothpaste, a mouthwash, a lotion, a gel, a patch, a spray, etc.

In another aspect, the present disclosure provides a method for preparing the Janus microparticle, which comprises:

(1) a process of synthesizing a polystyrene particle by dispersion polymerization;

(2) a process of dispersing the polystyrene particle in a mixture solvent of an alcohol and water;

(3) a process of swelling the polystyrene particle by absorbing a tetradecyl acrylate monomer into the polystyrene particle by adding the tetradecyl acrylate monomer to the mixture solvent; and

(4) a process of polymerizing the tetradecyl acrylate by photopolymerization and inducing phase separation.

In an exemplary embodiment, the dispersion polymerization in the process (1) may be performed in the presence of a compound for forming a hydrophilicity-inducing group on the surface of the polystyrene particle.

In an exemplary embodiment, the compound for forming a hydrophilicity-inducing group may be one or more selected from a group consisting of poly(vinyl alcohol), polyvinylpyrrolidone, polyethyleneimine or poloxamer. The poloxamer may be poloxamer 407 or a poly(ethylene oxide)-poly(propylene oxide)-poly(ethylene oxide) (PEO-PPO-PEO) triblock copolymer.

In an exemplary embodiment, the method may further include, after the process (4), (5) a process of forming a hydrophilic material coating layer by binding a silica nanoparticle to the hydrophilicity-inducing group.

In an exemplary embodiment, the mixture solvent in the process (2) may be a mixture of a C₁-C₆ alcohol and water at a volume ratio of 4:1-1:4. In another exemplary embodiment, the C₁-C₆ alcohol may be specifically ethanol. In another exemplary embodiment, the volume ratio may be 1-4:1-4, specifically 3:2.

In an exemplary embodiment, the process (3) may be performed by adding one or more of a crosslinking agent and a photopolymerization initiator. The crosslinking agent may include ethylene glycol dimethacrylate (EGDMA) and the photopolymerization initiator may include 1-hydroxycyclohexyl phenyl ketone.

With the preparation method, a Janus microparticle wherein clear phase separation is achieved can be prepared in large scale. Whereas the existing Janus particle has the problem that the degree of phase separation is indefinite and large-scale production is difficult, the present disclosure allows for the preparation of a Janus microparticle wherein clear phase separation is achieved in large scale (see FIG. 2 and FIG. 4).

In another aspect, the present disclosure provides a method for controlling the structure of an amphiphilic microparticle, wherein

the amphiphilic microparticle is prepared by a method comprising:

(1) synthesizing a polystyrene particle by dispersion polymerization;

(2) dispersing the polystyrene particle in a mixture solvent of an alcohol and water;

(3) swelling the polystyrene particle by absorbing an alkyl acrylate monomer into the polystyrene particle by adding the alkyl acrylate monomer to the mixture solvent; and

(4) polymerizing the alkyl acrylate by photopolymerization and inducing phase separation, and

the structure of the microparticle is controlled by one or more of:

changing the number of alkyl carbons in the alkyl acrylate monomer;

changing the mixture solvent; and

changing the swelling ratio of the polystyrene particle.

In an exemplary embodiment, the number of alkyl carbons may be changed in a range of 5-20. The number of alkyl carbons may be specifically 6, 12, 14 or 16, more specifically 14. And, the alkyl acrylate may include lauryl methacrylate.

In an exemplary embodiment, the mixture solvent may be changed by changing the volume ratio of a C₁-C₆ alcohol and water in a range of 4:1-1:4. In another exemplary embodiment, the C₁-C₆ alcohol may be specifically ethanol. In another exemplary embodiment, the volume ratio may be 1-4:1-4, specifically 3:2.

In the present disclosure, the swelling ratio refers to a second domain/first domain weight ratio (w/w) in the microparticle. The swelling ratio may be changed by controlling various conditions such as the amount of the monomer, the solvent, temperature, time, etc. when the particle is swollen by including the alkyl acrylate monomer in the polymerized polystyrene.

In an exemplary embodiment, the swelling ratio may be changed such that the degree of Janusity is 0.25-0.75. In another exemplary embodiment, the swelling ratio may be changed such that the degree of Janusity is 0.25 or greater, 0.3 or greater, 0.35 or greater, 0.37 or greater, 0.4 or greater, 0.45 or greater, 0.5 or greater, 0.55 or greater, 0.6 or greater or 0.7 or greater or 0.75 or smaller, 0.7 or smaller, 0.6 or smaller, 0.55 or smaller, 0.5 or smaller, 0.45 or smaller, 0.4 or smaller, 0.37 or smaller, 0.35 or smaller or 0.3 or smaller, specifically 0.45-0.55.

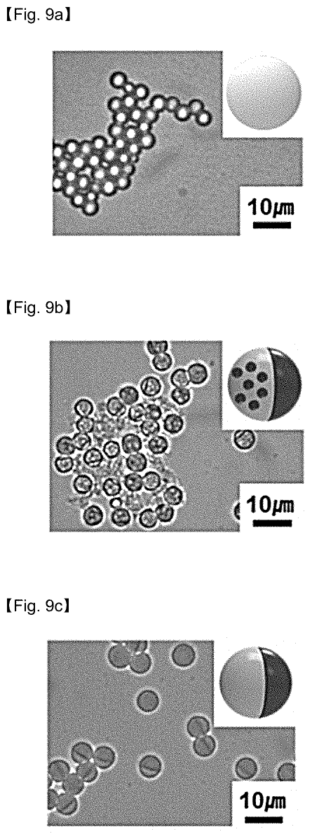

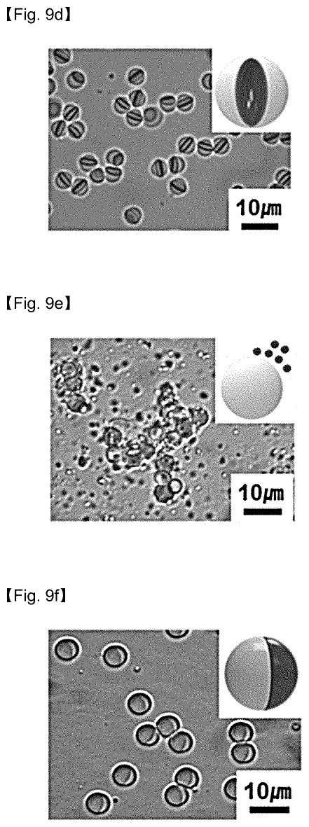

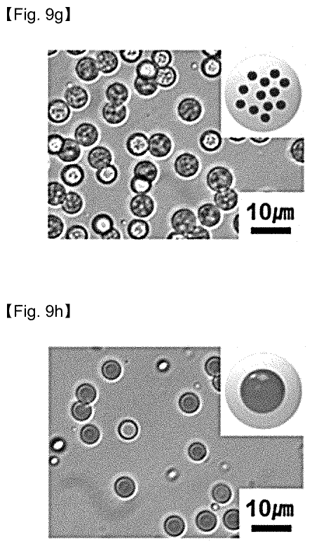

The degree of phase separation can be precisely controlled by changing the number of alkyl carbons in the alkyl acrylate monomer or by changing the alcohol:water volume ratio of the mixture solvent (see FIGS. 9a-9h).

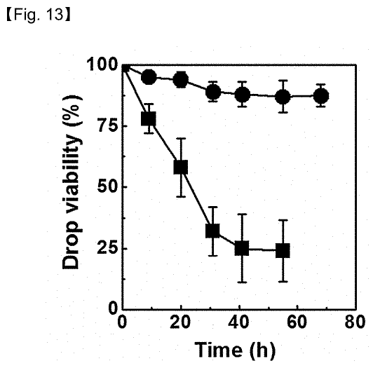

In addition, because the degree of Janusity can be precisely controlled by changing the swelling ratio (see FIGS. 10a-10c), the contact angle during interfacial assembly (FIG. 12) and the retention time of the Pickering emulsion (FIG. 13) can be improved remarkably.

Hereinafter, the present disclosure will be described in detail through examples. However, the following examples are for illustrative purposes only and it will be apparent to those of ordinary skill in the art that the scope of the present disclosure is not limited by the examples.

### <Example 1> Preparation of Janus Microparticle

To prepare a Janus microparticle of the present disclosure, styrene, polyvinylpyrrolidone (PVP, Mₙ=40,000 g mol⁻¹), anhydrous ethanol, poly(vinyl alcohol) (PVA, M_{w}=13,000-23,000 g mol⁻¹, 87-89% hydrolyzed), ethylene glycol dimethacrylate (EGDMA, 98%), 1-hydroxycyclohexyl phenyl ketone (Irgacure 184, 99%), hexyl acrylate (98%), dodecyl acrylate (96%), 9-vinylanthracene (VA) and poloxamer 407 (Pluronic F-127) were purchased from Sigma Aldrich (USA). 2,2′-Azobis(isobutyronitrile) (AIBN, 98%) was purchased from Junsei (Japan), tetradecyl acrylate (TA) and hexadecyl acrylate were purchased from TCI (Japan) and silica nanoparticles (KE-P10, KE-P30) were acquired from Nippon Shokubai (Japan). Deionized distilled water was used as water.

A bright-field microscope (Axio Vert. A1, Carl Zeiss, Germany) was used to observe each particle. The Janus phase of the particle was examined with a fluorescence microscope (Axio Vert. A1, Carl Zeiss, Germany). In this case, 9-vinylanthracene (0.1 wt %, Aldrich) as a fluorescence probe was copolymerized with a polystyrene polymer. The morphology of each particle was observed with a scanning electron microscope (SEM, S-4800, Hitachi, Japan) and the diameter was determined from analysis of the electron microscopic image. For this analysis, more than 100 particles were analyzed and the average was taken. The chemical property of the particle surface was analyzed with an X-ray photoelectron spectrometer (XPS, Theta Probe, Thermo Fisher Scientific, USA).

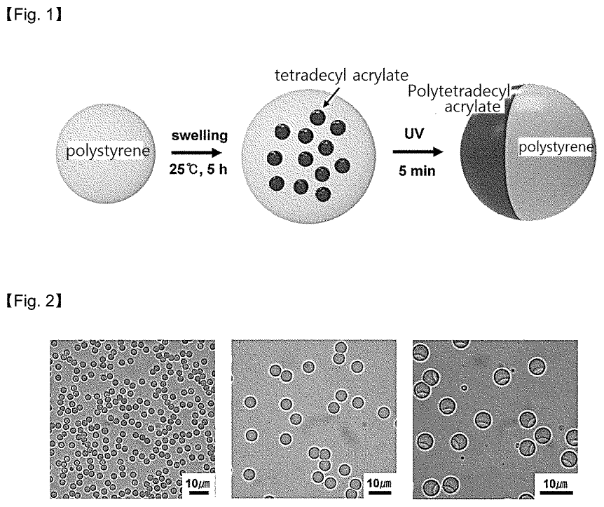

The Janus microparticle according to the present disclosure was prepared as shown in FIG. 1.

First, a polystyrene particle with a diameter calculated for an equivalent sphere of 3 μm was prepared by dispersion polymerization.

Specifically, 5 mL of styrene, 1.0 g of polyvinylpyrrolidone (PVP) and 0.05 g of AIBN were dissolved in anhydrous ethanol (50 mL, 200 proofs) in a 100-mL round-bottom flask. Nitrogen was purged for 5 minutes to remove oxygen during the reaction. Then, polymerization was carried out at 70° C. in an oil bath while stirring at 60 rpm for 48 hours. After the polymerization, polystyrene particle was washed repeatedly with ethanol and an ethanol/water mixture (1:1, v/v) by centrifugation to remove residual monomer and additive. The polystyrene particle was stored in an ethanol/water mixture (2/1, v/v). The concentration of the particle was set to 10 wt %.

The first image in FIG. 2 is the bright-field microscopic image of the polystyrene particle. It was confirmed from X-ray photoelectron spectroscopy (XPS) analysis that polyvinylpyrrolidone was covalently bonded on the surface of the polystyrene (FIG. 8). Because the polyvinylpyrrolidone (PVP) was grafted onto the surface of the polystyrene during the dispersion polymerization of the polystyrene (PS), the polystyrene seed showed a high-intensity N peak (FIG. 8, a). After the polystyrene/poly(tetradecyl acrylate) Janus particle was prepared, the intensity of the N peak decreased because the polystyrene portion was decreased as compared to the entire particle (FIG. 8, b). Through this, it was confirmed that the polyvinylpyrrolidone was grafted onto the surface of the polystyrene.

Then, a monodisperse Janus microparticle was prepared using a tetradecyl acrylate monomer by swelling and photopolymerization.

Specifically, 0.1 g of the synthesized polystyrene particle was dispersed in an ethanol/water mixture solvent (5 mL, 3/2, v/v). To prevent aggregation, the dispersion was sonicated at room temperature for 30 minutes. To stabilize the particle, poloxamer 407 (Pluronic F-127, 2 wt %) and poly(vinyl alcohol) (PVA, 2 wt %) were added. Then, a mixture of a tetradecyl acrylate monomer (65 wt %), ethylene glycol dimethacrylate (EDGMA, 20 wt %) as a crosslinking agent and 1-hydroxycyclohexyl phenyl ketone (Irgacure 184, 15 wt %) as a photopolymerization initiator was added to the polystyrene particle dispersion. Then, swelling was carried out at room temperature for 6 hours while rotating at 50 rpm. The second image in FIG. 2 is the bright-field microscopic image of the swollen particle.

After the swelling was completed, phase separation was carried out by exposing the mixture to UV radiation (A=365 nm, JHC1-051S-V2, A&D, Korea) for 5 minutes. The prepared Janus microparticle was washed with ethanol/water (1/1, v/v) to remove excess monomer and additive.

The third image in FIG. 2 is the bright-field microscopic image of the phase-separated particle. It was confirmed that the particle was spherical from an electron microscopic image (FIG. 6, second image) and that the polystyrene portion and the poly(tetradecyl acrylate) portion were clearly phase-separated in one spherical particle from a fluorescence microscopic image (FIG. 6, first image, the bright portion is the polystyrene portion and the dark portion is the poly(tetradecyl acrylate) portion). A result of comparing the size of the polystyrene particle and the Janus microparticle is shown in FIG. 7.

### <Example 2> Bonding of Silica Nanoparticle

To give amphiphilicity to the Janus microparticle, a silica nanoparticle was hydrogen bonded onto the polyvinylpyrrolidone covalently bonded on the surface of the polystyrene (FIG. 3).

Specifically, 0.015 g of the polystyrene/poly(tetradecyl acrylate) Janus particle and 0.01 g of a silica nanoparticle were dispersed well respectively in an ethanol/water mixture solvent (2.5 mL, 1/1, v/v). Then, the silica nanoparticle dispersion was added dropwise to the Janus particle dispersion over 30 minutes while gently sonicating the mixture at room temperature. Then, the mixture was rotated at room temperature for 24 hours at a speed of 50 rpm. To remove remnant silica nanoparticle, the mixture was repeatedly centrifuged using an ethanol/water mixture solution (1/1, v/v). The produced amphiphilic Janus particle was stored in water at room temperature.

The bonded silica nanoparticle had a diameter of 100 nm or 300 nm. Electron microscopic images of the prepared amphiphilic Janus particles are shown in FIG. 4.

### <Example 3> Preparation of Pickering Emulsion

A Pickering emulsion was prepared using the silica particle-coated amphiphilic Janus microparticle.

Specifically, 1 wt % of the silica particle-coated amphiphilic Janus microparticle was finely dispersed in water by sonicating at room temperature for 5 minutes. Then, 10 vol % of hexadecane was added to the Janus particle dispersion. The mixture was then vortexed for 10 seconds, which produced a Janus particle-stabilized Pickering emulsion. The amphiphilic Janus particle of the present disclosure was readily wet by a compatible liquid phase (FIG. 5).

### <Test Example 1> Control of Morphology of Microparticle

Particles of various morphologies can be prepared by changing the monomer used in the examples when performing the swelling and photopolymerization.

Specifically, particles were prepared as described in the examples using hexyl acrylate, dodecyl acrylate, tetradecyl acrylate or hexadecyl acrylate as the monomer and their morphologies were observed using a bright-field microscope. As a result, particles of various morphologies as shown in FIG. 9a (hexyl acrylate), FIG. 9b (dodecyl acrylate), FIG. 9c (tetradecyl acrylate) and FIG. 9d (hexadecyl acrylate) could be prepared. It was confirmed that a sandwich-shaped particle morphology could be produced when a monomer with a long alkyl chain length (C>14) was used.

Also, it was confirmed that particles of various morphologies could be prepared by performing the swelling and photopolymerization with different volume ratios of the ethanol/water mixture solvent, with the monomer fixed as tetradecyl acrylate.

Specifically, when the swelling and photopolymerization were performed using tetradecyl acrylate as the monomer and using an ethanol/water mixture solvent with a volume ratio of 4/1, 3/2, 2/3 and 1/4, respectively, different particle morphologies were produced as shown in FIG. 9e (4/1), FIG. 9f (3/2), FIG. 9g (2/3) and FIG. 9h (1/4).

### <Test Example 2> Control of Degree of Janusity of Janus Microparticle

It was confirmed that the degree of Janusity of the Janus microparticle according to the present disclosure can be precisely controlled by controlling the swelling ratio of the particle.

Specifically, the degree of Janusity is defined as D/D₀, where D is the shorter diameter of the poly(tetradecyl acrylate) (PTA) portion of the particle excluding the polystyrene (PS) portion and D₀ is the whole diameter of the particle (see FIG. 10c). When D/D₀ was 0.25, the morphology of the Janus particle was as shown in FIG. 10a. And, when D/D₀ was 0.5, the morphology of the Janus particle was as shown in FIG. 10b. As can be seen from FIG. 10c, it was confirmed that the degree of Janusity could be tuned in a range of 0.25-0.5 by controlling the swelling ratio (PTA/PS, w/w). When D/D₀ was smaller than 0.25, irregular phase separation was observed. And, when D/D₀ was greater than 0.5, the monomer swelling did not proceed uniformly.

### <Test Example 3> Evaluation of Interfacial Assembly of Janus Particle

In order to demonstrate the self-assembly ability of the Pickering emulsion at an oil-water interface, its interfacial assembly ability was evaluated.

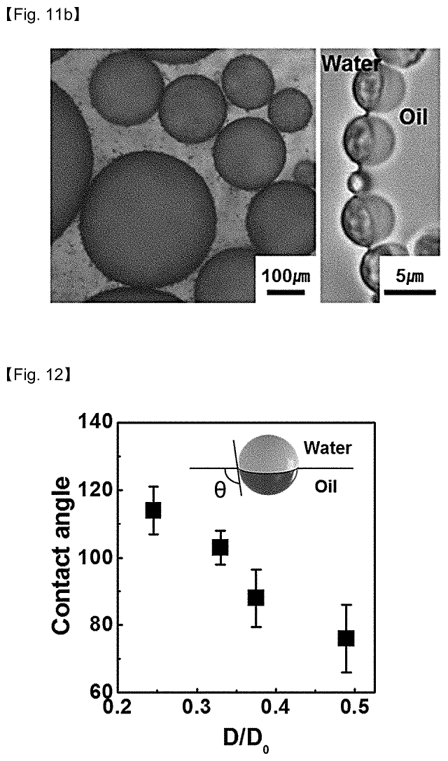

From the microscopic images of the Pickering emulsions prepared in the examples (FIGS. 11a and 11b), it was confirmed that a Pickering emulsion (oil-in-water, O/W) was formed with the side coated with hydrophilic silica contacting with water and the hydrophobic PTA side contacting with oil. It was also confirmed that the degree of Janusity D/D₀ of the second domain with respect to the entire Janus microparticle determines the contact angle at the assembled interface (FIG. 12) and that whether the particle will be W/0 or 0/W can be determined thereby. When the degree of Janusity was equal to or greater than 0.25 and smaller than 0.37, the particle was water-in-oil (w/o) type. And, when it was equal to or greater than 0.37 and smaller than 0.75, the particle was oil-in-water (o/w) type (FIG. 12).

The unique wetting behavior of the amphiphilic Janus particle plays a critical role in the structural stability of the Pickering emulsion. The adhesion energy (E) of the Pickering emulsion is expressed by E=πa²γ (1±cos θ)², where a is the radius of the particle, γ is the interfacial tension and θ is the contact angle. If the radius of the particle and the interfacial tension are the same, the adhesion energy increases as the contact angle is smaller. Therefore, a stable Pickering emulsion system can be obtained when the degree of Janusity D/D₀ is close to 0.5.

Indeed, the viability of the Pickering emulsion drop with time was remarkably improved when the degree of Janusity D/D₀ was 0.5 (circles in FIG. 13) as compared to when D/D₀=0.25 (squares in FIG. 13).

While the exemplary embodiments have been shown and described, it will be understood by those skilled in the art that various changes in form and details may be made thereto without departing from the spirit and scope of this disclosure as defined by the appended claims.

1. A Janus microparticle comprising:
a first domain comprising polystyrene; and
a second domain comprising poly(tetradecyl acrylate).

2. The Janus microparticle according to claim 1, wherein a hydrophilicity-inducing group is covalently bonded on the surface of the polystyrene of the first domain.

3. The Janus microparticle according to claim 2, wherein the first domain comprises:
a core comprising polystyrene; and
a hydrophilic material coating layer coated on the core.

4. The Janus microparticle according to claim 3, wherein the hydrophilic material coating layer comprises a hydrophilic material bonded to the hydrophilicity-inducing group covalently bonded on the surface of the polystyrene.

5. The Janus microparticle according to claim 2, wherein the hydrophilicity-inducing group comprises one or more selected from a group consisting of poly(vinyl alcohol), polyvinylpyrrolidone and poloxamer.

6. The Janus microparticle according to claim 4, wherein the hydrophilic material comprises a silica nanoparticle.

7. The Janus microparticle according to claim 1, wherein the Janus microparticle has a degree of Janusity of the second domain with respect to the entire particle of 0.25-0.75.

8. The Janus microparticle according to claim 1, wherein the Janus microparticle has a diameter calculated for an equivalent sphere ranging from 1 micrometer to 100 micrometers.

9. An emulsion composition comprising the Janus microparticle according to claim 1.

10. The emulsion composition according to claim 9, wherein the emulsion is a Pickering emulsion.

11. The emulsion composition according to claim 9, wherein the emulsion is a water-in-oil (w/o) emulsion when a degree of Janusity of a second domain with respect to the entire Janus microparticle is equal to or greater than 0.25 and smaller than 0.37 and is an oil-in-water (o/w) emulsion when the degree of Janusity is equal to or greater than 0.37 and smaller than 0.75.

12. (canceled)

13. A method for preparing the Janus microparticle according to claim 1, which comprises the processes:
(1) synthesizing a polystyrene particle by dispersion polymerization;
(2) dispersing the polystyrene particle in a mixture solvent of an alcohol and water;
(3) swelling the polystyrene particle by absorbing a tetradecyl acrylate monomer into the polystyrene particle by adding the tetradecyl acrylate monomer to the mixture solvent; and
(4) polymerizing the tetradecyl acrylate by photopolymerization and inducing phase separation.

14. The method for preparing the Janus microparticle according to claim 13, wherein the dispersion polymerization in the process (1) is performed in the presence of a compound for forming a hydrophilicity-inducing group on the surface of the polystyrene particle.

15. The method for preparing the Janus microparticle according to claim 13, which further comprises, after the process (4), (5) forming a hydrophilic material coating layer by binding a silica nanoparticle to the hydrophilicity-inducing group.

16. The method for preparing the Janus microparticle according to claim 13, wherein the mixture solvent in the process (2) is a mixture of a C₁-C₆ alcohol and water at a volume ratio of 4:1-1:4.

17. The method for preparing the Janus microparticle according to claim 13, wherein the process (3) is performed by adding one or more of a crosslinking agent and a photopolymerization initiator.

18. A method for controlling the structure of an amphiphilic microparticle,
wherein the amphiphilic microparticle is prepared by a method comprising:
(1) synthesizing a polystyrene particle by dispersion polymerization;
(2) dispersing the polystyrene particle in a mixture solvent of an alcohol and water;
(3) swelling the polystyrene particle by absorbing an alkyl acrylate monomer into the polystyrene particle by adding the alkyl acrylate monomer to the mixture solvent; and
(4) polymerizing the alkyl acrylate by photopolymerization and inducing phase separation, and the structure of the microparticle is controlled by one or more of:
changing the number of alkyl carbons in the alkyl acrylate monomer;
changing the mixture solvent; and
changing the swelling ratio of the polystyrene particle.

19. The method for controlling the structure of an amphiphilic microparticle according to claim 18, wherein the number of alkyl carbons is changed in a range of 5-20.

20. The method for controlling the structure of an amphiphilic microparticle according to claim 18, wherein the mixture solvent is changed by changing the volume ratio of a C₁-C₆ alcohol and water in a range of 4:1-1:4.

21. The method for controlling the structure of an amphiphilic microparticle according to claim 18, wherein the swelling ratio is changed such that the degree of Janusity of a second domain with respect to the entire Janus microparticle is 0.25-0.75.
